(19) United States
(12) Patent Application Publication
(10) Pub. No.: US 2023/0334132 A1
(43) Pub. Date: Oct. 19, 2023

(54) HEAD MOUNTED INFORMATION PROCESSING DEVICE, AUTHENTICATION SYSTEM, AND AUTHENTICATION METHOD

(71) Applicant: MAXELL, LTD., Kyoto (JP)

(72) Inventors: Mayumi NAKADE, Kyoto (JP); Nicholas Simon WALKER, London (GB); Jan Jasper VAN DEN BERG, London (GB); Yasunobu HASHIMOTO, Kyoto (JP); Osamu KAWAMAE, Kyoto (JP)

(21) Appl. No.: 18/026,906

(22) PCT Filed: Sep. 18, 2020
(86) PCT No.: PCT/JP2020/035610

§ 371 Date: Mar. 17, 2023

(51) Int. Cl. G06F 21/32; G02B 27/01

(52) U.S. Cl. CPC G06F 21/32; G02B 27/017

(57) ABSTRACT

A head mounted information processing device, an authentication system, and an authentication method capable of achieving an improvement in security are provided. Thus, a head mounted information processing device 1 includes a display 45 configured to display an image, a microphone 50 configured to collect a voice of a user and output an audio data signal, a biometric authentication sensor 30 configured to acquire tomographic data of a head of the user, and a controller (20) configured to control the head mounted information processing device 1. The controller (20) authenticates the user based on the tomographic data acquired by the biometric authentication sensor 30 during an authentication period in which the user is uttering a passcode.

### TECHNICAL FIELD

The present invention relates to a head mounted information processing device, an authentication system, and an authentication method, and relates to, for example, a technology for authenticating a user of a head mounted information processing device.

### BACKGROUND ART

Patent Document 1 discloses a personal authentication device capable of improving the accuracy of personal authentication. Specifically, the personal authentication device includes an image authentication unit that performs face authentication, a voice authentication unit that performs voice authentication, an ID authentication unit that authenticates a keyword or an ID number, and a fingerprint authentication unit that performs fingerprint authentication, and the personal authentication device performs personal authentication by using at least two units in combination from among the authentication units.

### RELATED ART DOCUMENTS

### Patent Documents

Patent Document 1: Japanese Unexamined Patent Application Publication No. 2000-259828

### SUMMARY OF THE INVENTION

### Problems to be Solved by the Invention

A head mounted information processing device is referred to also as a head mounted display (HMD) and is capable of displaying augmented reality (AR) information or virtual reality (VR) information on its display while being mounted on a head of a user. For example, at the time of some sort of work with such an HMD, for prevention of leakage of various types of confidential information acquirable through the HMD, authentication of whether a user to use the HMD is a legitimate user is required.

Meanwhile, in recent years, along with the development of artificial intelligence (AI), the technology called deepfake has been acknowledged as a problem. Use of such deepfake technology may enable, for example, a malicious user to impersonate the voice of a legitimate user. As a result, for example, a malicious user wearing an HMD is enabled to impersonate a legitimate user to succeed in voice authentication.

Thus, as disclosed in Patent Document 1, combining a plurality of authentication methods different in type is conceivable. However, an increase in the number of authentication methods to be combined is likely to cause intricate processing or an increase in cost. Further, since each individual authentication method disclosed in Patent Document 1 alone may be insufficient to prevent impersonation, even a plurality of authentication methods in combination cannot necessarily ensure adequate security. Furthermore, for example, like fingerprint authentication, there is an authentication method unsuitable for application to an HMD.

The present invention has been made in consideration of the above, and one of the objects of the present invention is to provide a head mounted information processing device, an authentication system, and an authentication method capable of achieving an improvement in security.

The above and other objects and novel features of the present invention will be clarified from the descriptions in this specification and the accompanying drawings.

### MEANS FOR SOLVING THE PROBLEMS

According to an embodiment of the present invention, provided is a head mounted information processing device configured to provide various types of information to a user in a visual or auditory manner while being mounted on a head of the user, the head mounted information processing device includes: a display configured to display an image; a microphone configured to collect a voice of the user and output an audio data signal; a biometric authentication sensor configured to acquire tomographic data of the head of the user; and a controller configured to control the head mounted information processing device, and the controller is configured to authenticate the user based on the tomographic data acquired by the biometric authentication sensor during an authentication period in which the user is uttering a passcode.

### Effects of the Invention

The effect obtained by the typical embodiment of the invention disclosed in this application will be briefly described below. That is, it is possible to achieve an improvement in security.

### BRIEF DESCRIPTIONS OF THE DRAWINGS

FIG. 1 is a schematic diagram illustrating an exemplary configuration of main parts in an authentication system according to the first embodiment of the present invention;

FIG. 2(a) and FIG. 2(b) are schematic diagrams illustrating respectively different exemplary configurations of a biometric authentication sensor in FIG. 1;

FIG. 3 is a block diagram illustrating an exemplary detailed configuration of a head mounted information processing device in FIG. 1;

FIG. 4 is a flowchart illustrating an exemplary authentication method using the authentication system in FIG. 1 and the head mounted information processing device in FIG. 3;

FIG. 5 is a supplementary view for describing part of the processing details in FIG. 4;

FIG. 6 is a supplementary view for describing part of the processing details in FIG. 4;

FIG. 7 is a block diagram illustrating an exemplary configuration of main parts of an authentication server in FIG. 1;

FIG. 8 is a sequence diagram illustrating exemplary processing details in a case where the authentication system in FIG. 1 is applied to a conference system, in an authentication method according to the second embodiment of the present invention;

FIG. 9 is a schematic diagram illustrating an exemplary configuration in a case of application to a conference system, in an authentication system according to the second embodiment of the present invention;

FIG. 10 is a sequence diagram illustrating exemplary processing details in the conference system (authentication system) in FIG. 9; and

FIG. 11 is a schematic diagram illustrating an exemplary configuration and an exemplary operation of main parts in a case of application to a shopping system, in an authentication system according to the third embodiment of the present invention.

### DETAILED DESCRIPTION OF PREFERRED EMBODIMENTS

Hereinafter, embodiments of the present invention will be described in detail with reference to the drawings. Note that the same members are denoted by the same reference characters in all the drawings for describing the embodiments, and repetitive description thereof will be omitted in principle.

### FIRST EMBODIMENT

<<Outline of Authentication System>>

FIG. 1 is a schematic diagram illustrating an exemplary configuration of main parts in an authentication system according to the first embodiment of the present invention. An authentication system 10 illustrated in FIG. 1 includes a head mounted information processing device (abbreviated to as an HMD in the specification) 1, a wireless router 11, a communication network 12, an authentication server 13, and a storage device 14. The HMD 1 provides various types of information to a user 5 in a visual or auditory manner while being mounted on a head of the user 5. The communication network 12 is, for example, the Internet or Ethernet (registered trademark).

The wireless router 11 is connected to the communication network 12 and performs wireless communication with the HMD 1, thereby relaying the communication of the HMD 1 through the communication network 12. The authentication server 13 is connected to the communication network 12 and performs predetermined processing involved in authentication. The storage device 14 stores various types of registration data necessary for authentication. The storage device 14 may be provided inside the authentication server 13.

Here, the HMD 1 includes a display 45 and biometric authentication sensors 30. The display 45 displays a predetermined image. Though described in detail below, the biometric authentication sensors 30 acquire tomographic data of the head of the user 5. The biometric authentication sensors 30 are provided at a plurality of positions where the head of the user 5 and the HMD 1 come in contact with each other. In this example, the HMD 1 is of a glasses type in shape. In this case, for example, the biometric authentication sensors 30 are provided at end tips in contact with the back side of ears and a pad in contact with the nose. However, for the HMD 1, various types in shape have been known such as a goggles type. The positions where the biometric authentication sensors 30 are to be provided and the number of the biometric authentication sensors 30 to be provided can be determined as appropriate in accordance with the shape of the HMD 1.

<<Outline of Biometric Authentication Sensor>>

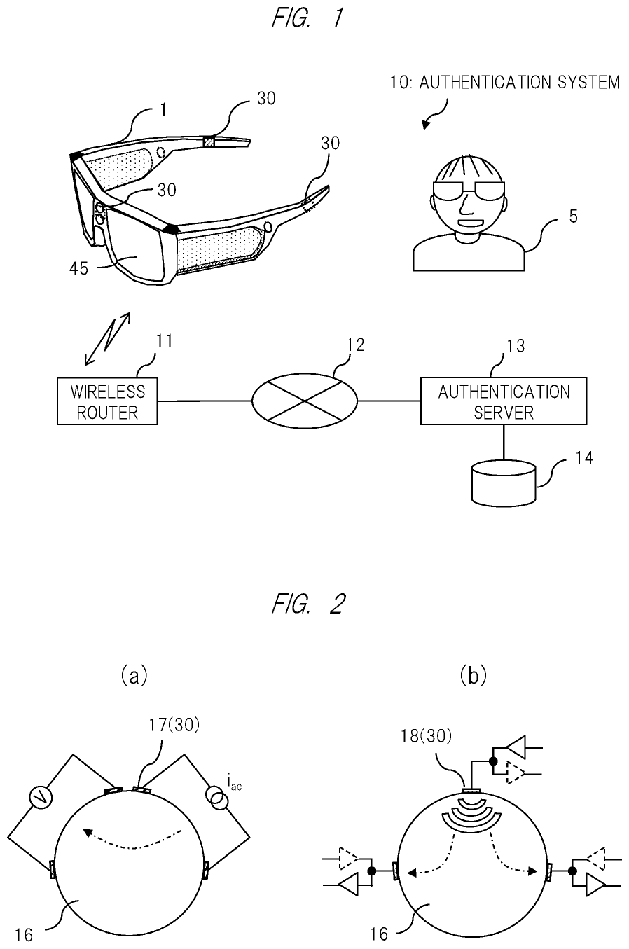

FIG. 2(a) and FIG. 2(b) are schematic diagrams illustrating respectively different exemplary configurations of the biometric authentication sensor in FIG. 1. The biometric authentication sensor 30 in FIG. 2(a) adopts the technology of electrical impedance tomography (EIT) and includes a plurality of electrodes 17 to acquire the distribution of electrical impedance in a head 16 as tomographic data. Electrical impedance tomography is a technology to measure the electrical impedance (conductivity) in the body and visualize tomographic data of the body (herein, tomographic data of the head 16).

Specifically, as illustrated in FIG. 2(a), of the two pairs of electrodes among the plurality of electrodes 17 provided at respectively different positions, one pair of electrodes is determined as an emitter serving as a signal source and the other pair of electrodes is determined as a receiver. The emitter generates AC current i_{ac} feeble (e.g., at a level of milliamperes (mA)) at a level of tens of kilohertz (kHz) to hundreds of kilohertz (kHz), and the receiver (one receiver in this example) measures voltage (V) corresponding to the AC current i_{ac}. Then, such processing is performed while sequentially changing the positional relationship between the emitter and the receiver, thereby enabling visualization of the distribution of electrical impedance (namely, tomographic data) in the head 16 surrounded by the plurality of electrodes 17.

The biometric authentication sensor 30 in FIG. 2(b) adopts the technology of acoustic interferometry and includes a plurality of ultrasonic transducers 18 to acquire an acoustic interference pattern in the head 16 as tomographic data. Acoustic interferometry is a technology to produce an acoustic interference pattern in the body with piezoelectric elements such as ultrasonic transducers 18 and visualize tomographic data of the body (herein, tomographic data of the head 16).

Specifically, as illustrated in FIG. 2(b), of the plurality of ultrasonic transducers 18 provided at respectively different positions, a single ultrasonic transducer 18 or a plurality of ultrasonic transducers 18 (in this example, a single ultrasonic transducer 18) is determined as an emitter serving as a signal source and the other ultrasonic transducers 18 are determined as receivers. For example, the emitter is driven with a pulse signal at tens of kilohertz (kHz) to emit an ultrasonic signal into the head 16, and each receiver receives an acoustic signal from inside of the head 16 corresponding to the ultrasonic signal. Then, such processing is performed while sequentially changing the positional relationship between the emitter and the receivers, thereby enabling visualization of an acoustic interference pattern (namely, tomographic data) in the head 16 surrounded by the plurality of ultrasonic transducers 18.

For example, tomographic data by electrical impedance tomography (EIT) is generally used for medical purposes. In this case, for example, a large number of electrodes 17 need to be arranged regularly in order to acquire tomographic data capable of specifying the detailed internal positions. Meanwhile, in the present embodiment, the tomographic data from the biometric authentication sensor 30 is used for personal identification at the time of authentication. In such a case, the electrodes 17 are not particularly required to be arranged regularly, and the number of electrodes 17 to be provided is not particularly limited as long as personal identification is possible.

<<Details of Head Mounted Information Processing Device (HMD)>>

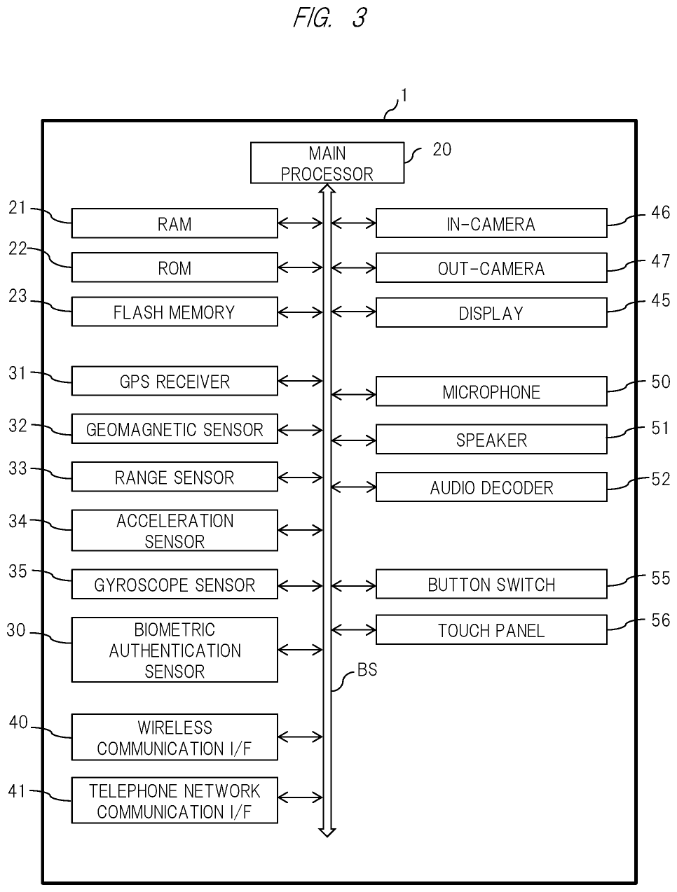

FIG. 3 is a block diagram illustrating an exemplary detailed configuration of the head mounted information processing device in FIG. 1. The HMD 1 in FIG. 3 includes various types of units mutually connected through a bus BS. The HMD 1 in FIG. 3 includes a main processor (controller) 20, a random access memory (RAM) 21, a read only memory (ROM) 22, and a flash memory 23 as the units for control system.

For example, the main processor (controller) 20 is composed of a single central processing unit (CPU), a plurality of CPUs, a single graphics processing unit (GPU), or a plurality of GPUs. The ROM 22 and the flash memory 23 each store, for example, a program for the main processor 20 or various types of parameter data. The main processor 20 executes the program to control the entirety of the HMD 1 through the bus BS. For example, the RAM 21 is used as a memory for work involved in program processing of the main processor 20.

The HMD 1 includes the biometric authentication sensor 30 described above, a global positioning system (GPS) receiver 31, a geomagnetic sensor 32, a range sensor 33, an acceleration sensor 34, and a gyroscope sensor 35 as the units for sensor system. The GPS receiver 31 detects the position of the HMD 1 based on radio waves from GPS satellites. The geomagnetic sensor 32 detects the magnetic force of the earth to detect the direction in which the HMD 1 faces. The range sensor 33 emits, for example, laser light to an object and detects the distance to the object based on time of flight (ToF). The acceleration sensor 34 detects the accelerations in the three axial directions of the HMD 1 to detect the motion, vibration, impact, and others of the HMD 1. The gyroscope sensor 35 detects the angular velocities around the three axes of the HMD 1 to detect the vertical, horizontal, and oblique attitudes of the HMD 1.

The HMD 1 includes a wireless communication interface 40 and a telephone network communication interface 41 as the units for communication system. The wireless communication interface 40 is typically a wireless LAN based on IEEE802.11. For example, the wireless communication interface 40 performs wireless communication with the authentication server 13 through the wireless router 11 and the communication network 12 in FIG. 1. For example, the telephone network communication interface 41 is an interface based on a standard such as 4th Generation (4G) or 5th Generation (5G) and performs wireless communication with a base station. For example, even in a case where the HMD 1 is out of the area of the wireless LAN, the HMD 1 can perform wireless communication with the authentication server 13 through the telephone network communication interface 41.

The HMD 1 includes the display 45, an in-camera 46, and an out-camera 47 as the units for visual system. The display 45 is, for example, a liquid crystal panel, and displays a predetermined image such as an AR image or a VR image. The in-camera 46 captures an image of an inner side of the HMD 1 (e.g., the user 5). The out-camera 47 captures an image of an outer side of the HMD 1.

The HMD 1 includes a microphone 50, a speaker 51, and an audio decoder 52 as the units for auditory system. The microphone 50 collects the voice of the user 5 and outputs an audio data signal. The speaker 51 converts an audio data signal generated in the HMD 1 into sound (sound wave) and outputs the sound to the user 5. For example, the audio decoder 52 performs processing to decode a coded audio data signal.

The HMD 1 includes a button switch 55 and a touch panel 56 as the units serving as a user interface. For example, the button switch 55 corresponds to remote control switches for various types of operations on a VR screen or an AR screen displayed on the display 45. For example, the touch panel 56 establishes a touch panel screen by VR or AR onto the display 45 and allows the user 5 to make predetermined operations based on the detection of the motion of a hand through the out-camera 47 or the like.

Here, though described in detail below, the main processor (controller) 20 causes the biometric authentication sensor 30 to acquire tomographic data during an authentication period in which the user 5 is uttering a passcode. Then, the main processor (controller) 20 authenticates the user 5 based on the tomographic data. Note that the HMD 1 may include various units in addition to the various types of units described above.

<<Details of Authentication Method>>

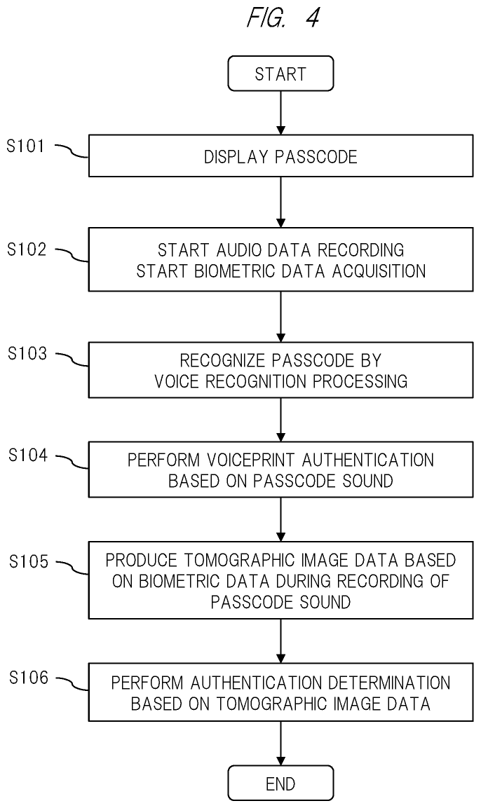

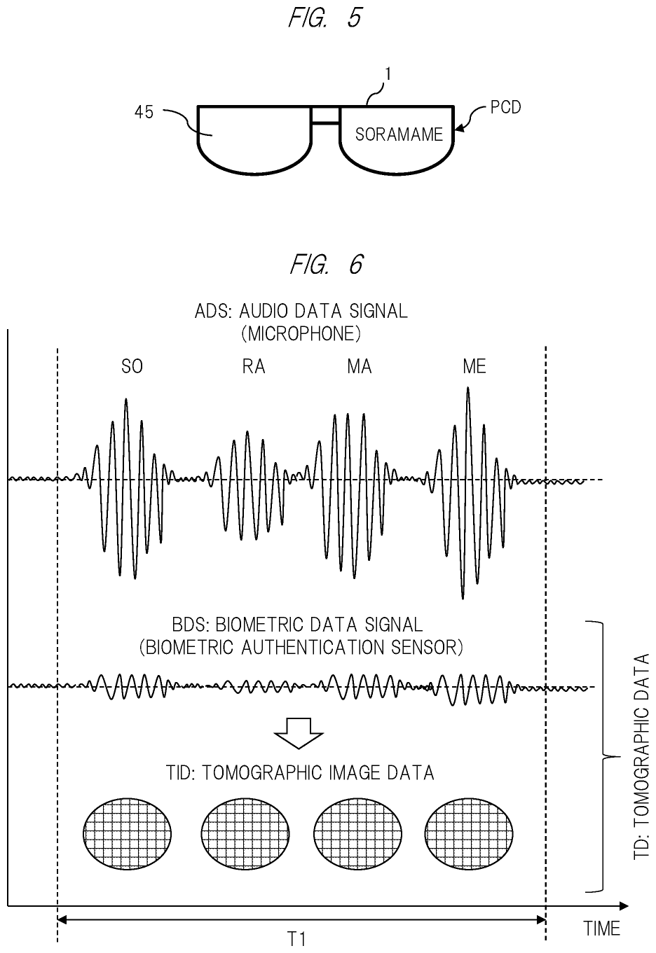

FIG. 4 is a flowchart illustrating an exemplary authentication method using the authentication system in FIG. 1 and the head mounted information processing device in FIG. 3. FIG. 5 and FIG. 6 are each supplementary views for describing part of the processing details in FIG. 4. In FIG. 4, first, the main processor (controller) 20 of the HMD 1 causes the display 45 to display a passcode (step S101). In the example of FIG. 5, “SORAMAME” is displayed as an exemplary passcode PCD on the display 45.

Subsequently, the main processor (controller) 20 starts recoding of an audio data signal with the microphone 50 (e.g., storage to the RAM 21) and acquisition and recoding of a biometric data signal with the biometric authentication sensor 30 (step S102). Meanwhile, after step S102, the user 5 utters the passcode PCD (“SORAMAME”) displayed in step S101. Along with this, the main processor (controller) 20 performs voice recognition of the passcode PCD uttered by the user 5 from the audio data signal output from the microphone 50 and recorded in the RAM 21 or the like (step S103).

FIG. 6 illustrates the audio data signal ADS from the microphone 50 which varies temporally, the biometric data signal BDS from the biometric authentication sensor 30 which varies temporally, and tomographic image data TID acquired from the biometric data signal BDS. The waveforms and others in FIG. 6 are expediently illustrated for description and thus are different from those in actual cases. The biometric data signal BDS corresponds to the signal acquired by each receiver described with reference to FIG. 2(a) or FIG. 2(b). Here, it is conceivable that the biometric data signal BDS varies as appropriate in accordance with the combination of the head structure of the user 5 and the utterance state of the user 5. Thus, the main processor (controller) 20 first performs voice recognition of the passcode PCD uttered by the user and detects, as an authentication period T1, the period in which the user 5 is uttering the passcode PCD.

Then, the main processor (controller) 20 converts the biometric data signal BDS acquired by the biometric authentication sensor 30 during the authentication period T1 into an image with a predetermined algorithm. In this way, the main processor (controller) 20 produces the tomographic image data TID which varies temporally. For example, the tomographic image data TID corresponds to two-dimensional image data indicating the distribution of electrical impedance in the head 16 or an acoustic interference pattern in the head 16 as described with reference to FIG. 2(a) or 2(b). In this specification, the tomographic image data TID or the biometric data signal BDS serving as a basis of the tomographic image data TID is referred to as tomographic data TD.

Referring back to FIG. 4, in step S104, for example, the authentication server 13 receives, from the HMD 1, the audio data signal ADS (passcode sound) in the authentication period T1 illustrated in FIG. 6. Then, the authentication server 13 performs voiceprint authentication based on the passcode sound. In the voiceprint authentication, as generally known, a spectrogram (relationship between time, frequency, and intensity of the signal component) of the audio data signal ADS is produced. Then, it is determined whether or not the authentication is successful based on the degree of similarity between the produced spectrogram and voiceprint data registered by the legitimate user in advance.

Subsequently, in step S105, the main processor (controller) or the authentication server 13 produces the tomographic image data TID based on the biometric data signal BDS in the authentication period T1 as illustrated in FIG. 6. In a case where the authentication server 13 produces the tomographic image data TID, the authentication server 13 is required to receive the biometric data signal BDS from the HMD 1.

Thereafter, in step S106, the authentication server 13 performs authentication determination based on the tomographic image data TID produced in step S105. Namely, the authentication server 13 collates the tomographic image data TID (tomographic data TD) with the registration data of the user 5 stored in advance in the storage device 14 and then transmits an authentication result acquired by the collation to the HMD 1. The HMD 1 authenticates the user 5 based on the authentication result (thus, the tomographic data TD) in step S106 and the result of the voiceprint authentication in step S104.

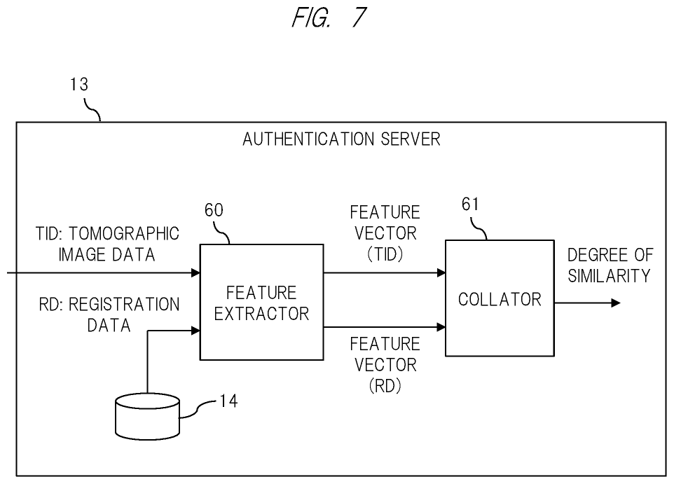

FIG. 7 is a block diagram illustrating an exemplary configuration of main parts of the authentication server in FIG. 1. The authentication server 13 in FIG. 7 includes a feature extractor 60 and a collator 61. The feature extractor 60 receives the tomographic image data TID or the registration data RD of the user stored in advance in the storage device 14 as an input and then extracts the feature with, for example, a trained deep learning model. The extracted feature is output in the form of a feature vector including information that the level of feature A is xx, the level of feature B is yy, and so on.

The collator 61 calculates the degree of similarity by collating the feature vector of the tomographic image data TID and the feature vector of the registration data RD. Then, the collator 61 determines the authentication result based on the calculated degree of similarity. For example, in a case where the degree of similarity is equal to or more than a previously determined threshold, the collator 61 determines the authentication result as authentication success. Also, in a case where the degree of similarity is less than the threshold, the collator 61 determines the authentication result as authentication failure. Note that the HMD 1 may perform the determination of whether or not the authentication is successful after collation. In this case, the authentication server 13 is required to determine the calculated degree of similarity as the authentication result.

Regarding the processing in step S104 and the processing in step S106 in FIG. 4, the HMD 1 may perform all the processing or the HMD 1 and the authentication server 13 may perform the allocated processing as appropriate. For example, in step S104, instead of the authentication server 13, the main processor (controller) 20 may perform the voiceprint authentication based on the voiceprint data of the legitimate user registered in advance in the ROM 22 or the flash memory 23.

Also, in step S106, instead of the authentication server 13, the main processor (controller) 20 may extract the feature from the tomographic image data TID. Namely, the feature extractor 60 (that is, the trained deep learning model or the like) illustrated in FIG. 7 may be implemented in the main processor (controller) 20. In this case, the HMD 1 transmits, to the authentication server 13, the feature vector of the tomographic image data TID from the feature extractor 60. Then, the authentication server 13 stores the feature vector of the registration data RD in the storage device 14 in advance, and collates the feature vector of the registration data RD and the feature vector of the tomographic image data TID from the HMD 1.

However, from the viewpoint of security management or the viewpoint of operation in a case where multiple users 5 use the HMD, it is often desirable that the voiceprint data for use in voiceprint authentication and the registration data RD for use in biometric authentication with the biometric authentication sensor 30 be managed by the authentication server 13. Therefore, from such viewpoints, desirably, the authentication server 13 includes at least the collator 61 in FIG. 7 (and a similar collator for voiceprint authentication).

Further, the passcode PCD is a character string to be selected or generated for each authentication. In a case where the HMD 1 generates the passcode PCD, the HMD 1 transmits, to the authentication server 13, the passcode PCD together with the feature vector.

### Main Effects in First Embodiment

As described above, use of the method according to the first embodiment typically enables an improvement in the security of the HMD. Specifically, since authentication can be performed based on the tomographic data TD that varies for each user and is likely to vary depending on the utterance details of the user, impersonation by the deepfake technology can be sufficiently prevented. In addition, use of voiceprint authentication enables further reliable prevention of impersonation. As another effect, an authentication method suitable to the usage mode of the HMD mounted on the head of the user can be realized. Namely, by simply providing the biometric authentication sensors 30 at predetermined locations on the HMD, a robust authentication method can be acquired.

Note that, in this example, voiceprint authentication and biometric authentication are performed based on the audio data signal ADS and the tomographic data TD in the authentication period T1, but only biometric authentication may be performed without voiceprint authentication. Alternatively, face authentication may be performed in addition to voiceprint authentication and biometric authentication. Specifically, the HMD 1 may perform further authentication by capturing a face image of the user 5 with the in-camera 46 and transmitting capture data of the face image or a feature extracted from the capture data to the authentication server 13, in addition to voiceprint authentication and biometric authentication.

### Second Embodiment

### Application Example to Conference System [1]

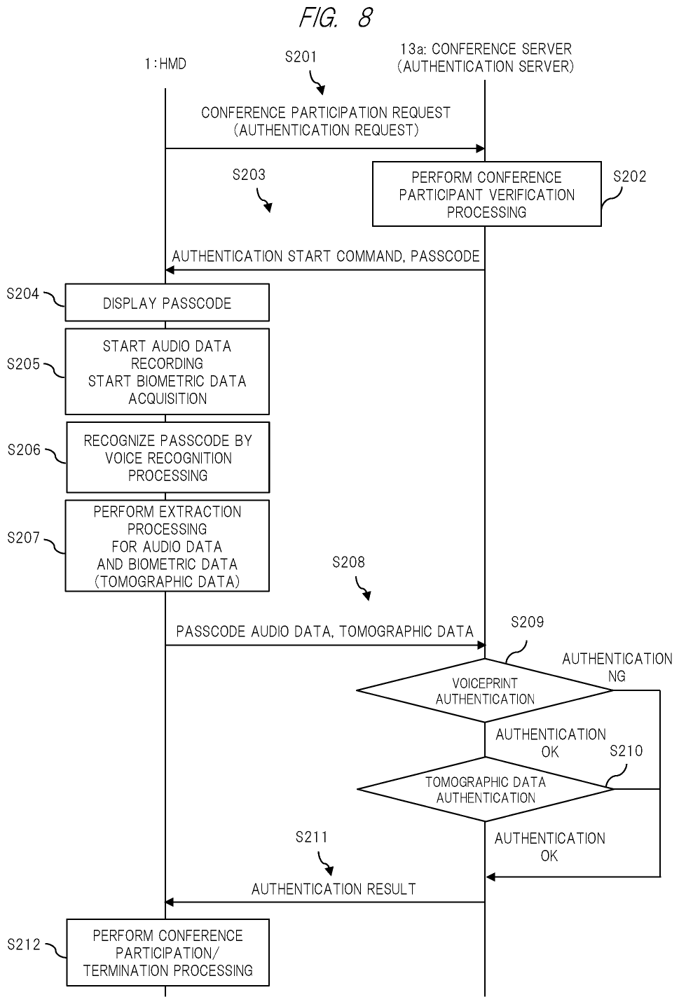

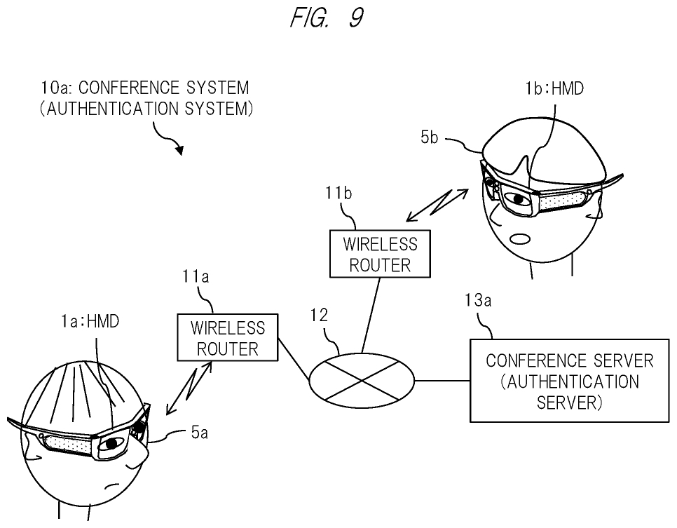

FIG. 8 is a sequence diagram illustrating exemplary processing details in a case where the authentication system in FIG. 1 is applied to a conference system, in an authentication method according to the second embodiment of the present invention. Herein, a case where the user 5 in FIG. 1 participates in a teleconference with the head mounted information processing device (HMD) 1 is assumed.

In FIG. 8, first, the HMD 1 transmits a conference participation request (in other words, an authentication request) to a conference server 13a (step S201). The conference participation request includes, for example, the identifier of the user 5 and the identifier of the conference. Also, the conference server 13a has the function of the authentication server 13 in FIG. 1. Subsequently, in response to the conference participation request in step S201, the conference server 13a verifies whether the user 5 is eligible to participate in the conference, based on previously registered conference information (step S202). Thereafter, the conference server 13a transmits an authentication start command and a passcode to the HMD 1 (step S203).

Next, the HMD 1 displays the passcode received in step S203 on the display 45 (step S204). Subsequently, similarly to steps S102 and S103 in FIG. 4, the HMD 1 starts recording of the audio data signal ADS and acquisition of the biometric data signal BDS (step S205) and performs voice recognition of the passcode from the audio data signal ADS (step S206).

Then, based on a recognition result in step S206, the HMD 1 performs extraction processing for the audio data signal ADS and the biometric data signal BDS (tomographic data TD) (step S207). Specifically, the HMD 1 determines the authentication period T1 described with reference to FIG. 6. In addition to this, the HMD 1 may perform processing to produce the tomographic image data TID from the biometric data signal BDS. Thereafter, the HMD 1 transmits the audio data signal ADS (that is, passcode audio data) and the tomographic data TD (biometric data signal BDS or tomographic image data TID) extracted in step S207 to the conference server 13a (step S208).

Subsequently, based on the passcode audio data received in step S208, the conference server 13a performs voiceprint authentication (step S209). In a case where the voiceprint authentication results in failure, the conference server 13a determines the authentication result as authentication failure without the processing in step S210. Meanwhile, in a case where the voiceprint authentication results in success, the conference server 13a performs biometric authentication based on the tomographic data TD received in step S208 (step S210). Then, in a case where the biometric authentication results in success, the conference server 13a determines the authentication result as authentication success. Also, in a case where the biometric authentication results in failure, the conference server 13a determines the authentication result as authentication failure.

Here, in the processing in step S210, in a case where the tomographic data TD received in step S208 is the biometric data signal BDS, the conference server 13a produces the tomographic image data TID, extracts a feature from the tomographic image data TID, and determines whether or not the authentication is successful based on the feature. Meanwhile, in a case where the tomographic data TD received in step S208 is the tomographic image data TID, the conference server 13a extracts a feature from the tomographic image data TID and determines whether or not the authentication is successful based on the feature.

Subsequently, in step S211, the conference server 13a transmits the authentication result determined in step S209 and S210 to the HMD 1. In a case where the authentication result received in step S211 is authentication success, the HMD 1 performs processing for participating in the conference. Also, in a case where the authentication result is authentication failure, the HMD 1 terminates the processing without participating in the conference (step S212).

Note that the passcode in step S203 is, for example, a passcode for challenge-response authentication. In this case, the conference server 13a stores, for example, voiceprint data and registration data RD for each passcode or for each character in advance into the storage device 14. Then, the conference server 13a randomly selects any of the plurality of passcodes in step S203 and performs authentication with the voiceprint data and the registration data RD corresponding to the selected passcode in steps S209 and S210.

In this manner, a further improvement can be made in security. Furthermore, the tomographic data TD transmitted in step S208 may be a feature vector extracted from the tomographic image data TID as described in the first embodiment.

### Application Example to Conference System [2]

FIG. 9 is a schematic diagram illustrating an exemplary configuration in a case of application to a conference system, in an authentication system according to the second embodiment of the present invention. A conference system (authentication system) 10a illustrated in FIG. 9 includes a plurality of head mounted information processing devices (HMDs) 1a and 1b and a plurality of wireless routers 11a and 11b, in addition to the communication network 12 similar to that in FIG. 1 and the conference server (authentication server) 13a. The HMDs 1a and 1b are mounted, respectively, on a plurality of users 5a and 5b. The wireless router 11a performs wireless communication with the HMD 1a to relay communication of the HMD 1a through the communication network 12, and the wireless router 11b performs wireless communication with the HMD 1b to relay communication of the HMD 1b through the communication network 12.

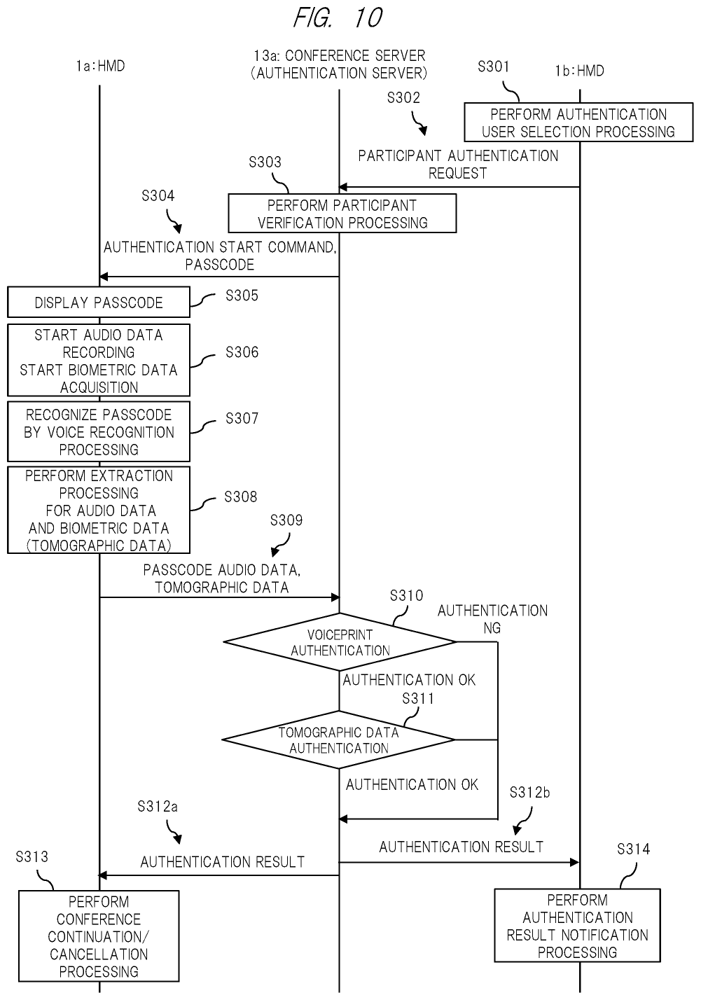

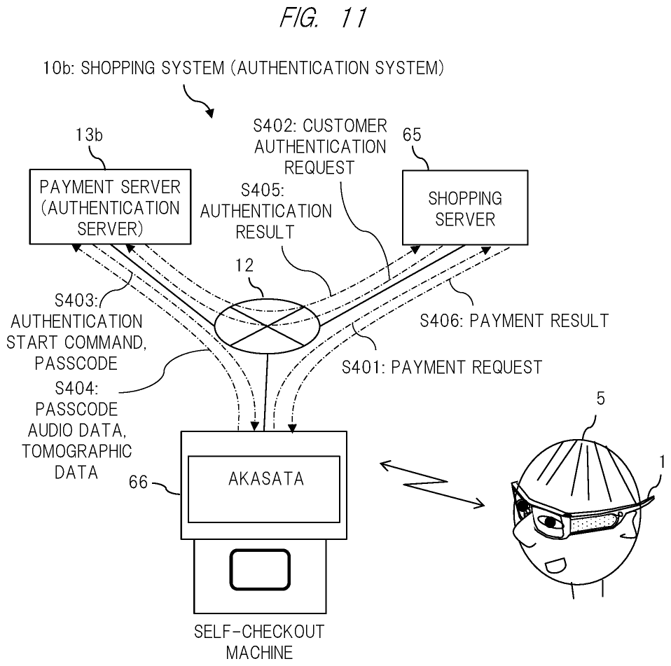

FIG. 10 is a sequence diagram illustrating exemplary processing details in the conference system (authentication system) in FIG. 9. Herein, unlike the case in FIG. 8, a case where the user 5b who is participating in a teleconference desires to reverify the eligibility of the user 5a who is participating in the teleconference is assumed. Namely, for example, a case where it is suspected that the user 5a has been replaced with anyone during the teleconference is assumed.

In FIG. 10, first, the HMD 1b prompts the user 5b to select the user 5a that the user 5b desires to reauthenticate (step S301). Subsequently, the HMD 1b transmits a participant authentication request to the conference server 13a (step S302). The participant authentication request includes, for example, the identifier of the selected user 5a. Next, in response to the participant authentication request in step S303, the conference server 13a verifies whether the user 5a is eligible for participation in the conference based on previously registered conference information (step S303). Thereafter, the conference server 13a transmits an authentication start command and a passcode to the HMD 1a (step S304).

Subsequently, in steps S305 to S309, the HMD 1a performs processing similar to the processing in steps S204 to S208 in FIG. 8. Briefly, the HMD 1a displays the received passcode on its display (step S305) and then starts recording of the audio data signal ADS and acquisition of the biometric data signal BDS (step S306). Further, the HMD 1a performs voice recognition of the passcode from the audio data signal ADS (step S307) and then performs extraction processing for the audio data signal ADS and the biometric data signal BDS (tomographic data TD) (step S308). Thereafter, the HMD 1a transmits the extracted audio data signal ADS (that is, passcode audio data) and tomographic data TD (biometric data signal BDS or tomographic image data TID) to the conference server 13a (step S309).

Next, in steps S310 and S311, the conference server 13a performs processing similar to the processing in steps S209 and S210 in FIG. 8. Briefly, the conference server 13a performs voiceprint authentication based on the received passcode audio data (step S310), and performs biometric authentication based on the received tomographic data TD in a case where the voiceprint authentication results in success (step S311). Then, in a case where both the voiceprint authentication and the biometric authentication result in success, the conference server 13a determines the authentication result as authentication success. Also, in a case where either the voiceprint authentication or the biometric authentication results in failure, the conference server 13a determines the authentication result as authentication failure.

Subsequently, the conference server 13a transmits the authentication result determined in steps S310 and S311 to the HMD 1a in step S312a and to the HMD 1b in step S312b. In a case where the authentication result received in step S312a is authentication success, the HMD 1a performs processing to continue the participation in the conference. Also, in a case where the authentication result is authentication failure, the HMD 1a performs processing to cancel the participation in the conference (step S313). Meanwhile, the HMD 1b performs processing to notify the user 5b of the authentication result received in step S312b through its display 45, speaker 51, or the like (step S314).

### Main Effects in Second Embodiment

As described above, similarly to the first embodiment, use of the method according to the second embodiment typically enables an improvement in the security of the HMD. In particular, it is possible to prevent a malicious user from participating in the teleconference. Namely, since a malicious user is required to pass the biometric authentication based on the tomographic data TD, it is difficult to impersonate a legitimate user even when deepfake technology is used. From these results, it is possible to prevent the leakage of various types of confidential information acquirable through the HMD during the conference.

### Third Embodiment

### Application Example to Shopping System

FIG. 11 is a schematic diagram illustrating an exemplary configuration and an exemplary operation of main parts in a case of application to a shopping system, in an authentication system according to the third embodiment of the present invention. A shopping system (authentication system) 10b illustrated in FIG. 11 includes the HMD 1, a payment server (authentication server) 13b, the communication network 12, a shopping server 65, and a self-checkout machine 66. The HMD 1 is mounted on the user (customer) 5.

The payment server 13b, the shopping server 65, and the self-checkout machine 66 are mutually connected through the communication network 12. The self-checkout machine 66 includes a wireless communicator that performs wireless communication with the HMD 1. The shopping server 65 is, for example, a server of a shopping company and manages the entirety of the shopping system 10b in addition to managing the self-checkout machine 66. The payment server (authentication server) 13b is, for example, a server of a credit-card company.

In such a configuration, first, the user (customer) 5 selects the items to be purchased and starts credit payment with the self-checkout machine 66. In response to this, the self-checkout machine 66 transmits a payment request to the shopping server 65 (step S401). The payment request includes, for example, the number of a credit card and a passcode. In response to the payment request in step S401, the shopping server 65 transmits a customer authentication request including, for example, the number of the credit card and the passcode to the payment server 13b (step S402).

In response to the customer authentication request in step S402, the payment server 13b identifies the customer and transmits an authentication start command and a separately generated passcode (in this example, “AKASATA”) to the self-checkout machine 66 (step S403). The self-checkout machine 66 displays the passcode in step S403 on its screen. Meanwhile, the user 5 utters the passcode displayed on the screen. In response to this, the HMD 1 performs processing similar to the processing in steps S205 to S207 in FIG. 8 and then transmits passcode audio data and tomographic data TD to the self-checkout machine 66, similarly to step S208. Also, the self-checkout machine 66 transmits the received passcode audio data and tomographic data TD to the payment server 13b (step S404).

Next, the payment server 13b performs voiceprint authentication based on the passcode audio data and biometric authentication based on the tomographic data TD similarly to steps S209 and S210 in FIG. 8, and transmits an authentication result to the shopping server 65 similarly to step S211 (step S405). In a case where the authentication result received in step S405 is authentication success as a payment result, the shopping server 65 causes the self-checkout machine 66 to complete the payment. Also, in a case where the authentication result is authentication failure, the shopping server 65 causes the self-checkout machine 66 to deny the payment (step S406).

### Main Effects in Third Embodiment

As described above, similarly to the first embodiment, use of the method according to the third embodiment typically enables an improvement in the security of the HMD. In particular, it is possible to prevent a malicious user from doing shopping illegally with someone's credit card. Namely, since a malicious user is required to pass the biometric authentication based on the tomographic data TD, it is difficult to impersonate a legitimate user even when deepfake technology is used.

Note that the present invention is not limited to the embodiments described above and includes various modifications. For example, the embodiments above have been described in detail in order to make the present invention easily understood, and the present invention is not necessarily limited to the embodiments having all of the described configurations. Also, part of the configuration of one embodiment may be replaced with the configuration of another embodiment, and the configuration of one embodiment may be added to the configuration of another embodiment.

Furthermore, another configuration may be added to part of the configuration of each embodiment, and part of the configuration of each embodiment may be eliminated or replaced with another configuration.

In addition, each configuration, function, processor, processing function, and the like described above may be realized by hardware by designing part or all of them by, for example, integrated circuits. Further, each configuration, function, and the like described above may be realized by software by interpreting and executing the program for realizing each function by the processor. Information such as programs, tables, and files for realizing each function can be stored in a memory, a storage device such as a hard disk or an SSD (Solid State Drive), or a storage medium such as an IC card, an SD card, or a DVD.

Also, the control lines and information lines that are considered to be necessary for explanation are illustrated, and all of the control lines and information lines in the product are not necessarily illustrated. In practice, it is safe to assume that almost all configurations are connected to each other.

### REFERENCE SIGNS LIST

1 Head mounted information processing device (HMD)
5, 5a, 5b User
10, 10a, 10b Authentication system
11, 11a, 11b Wireless router
12 Communication network
13, 13a, 13b Authentication server
14 Storage device
16 Head
17 Electrode
18 Ultrasonic transducer
20 Main processor (controller)
21 RAM
22 ROM
23 Flash memory
30 Biometric authentication sensor
31 GPS receiver
32 Geomagnetic sensor
33 Range sensor
34 Acceleration sensor
35 Gyroscope sensor
40 Wireless communication interface
41 Telephone network communication interface
45 Display
46 In-camera
47 Out-camera
50 Microphone
51 Speaker
52 Audio decoder
55 Button switch
56 Touch panel
60 Feature extractor
61 Collator
65 Shopping server
66 Self-checkout machine
ADS Audio data signal
BDS Biometric data signal
BS Bus
PCD Passcode
RD Registration data
T1 Authentication period
TD Tomographic data
TID Tomographic image data

1. A head mounted information processing device configured to provide various types of information to a user in a visual or auditory manner while being mounted on a head of the user, the head mounted information processing device comprising:
a display configured to display an image;
a microphone configured to collect a voice of the user and output an audio data signal;
a biometric authentication sensor configured to acquire tomographic data of the head of the user; and
a controller configured to control the head mounted information processing device,
wherein the controller authenticates the user based on the tomographic data acquired by the biometric authentication sensor during an authentication period in which the user is uttering a passcode.

2. The head mounted information processing device according to claim 1,
wherein the biometric authentication sensor includes a plurality of electrodes to acquire a distribution of electrical impedance in the head as the tomographic data.

3. The head mounted information processing device according to claim 1,
wherein the biometric authentication sensor includes a plurality of ultrasonic transducers to acquire an acoustic interference pattern in the head as the tomographic data.

4. The head mounted information processing device according to claim 1,
wherein the controller extracts a feature from the tomographic data and authenticates the user based on data of the feature.

5. The head mounted information processing device according to claim 1,
wherein, after causing the display to display the passcode, the controller detects the authentication period by performing voice recognition of the passcode uttered by the user from the audio data signal.

6. The head mounted information processing device according to claim 1,
wherein the controller authenticates the user based on a result of voiceprint authentication based on the audio data signal in the authentication period and the tomographic data.

7. An authentication system comprising:
a head mounted information processing device configured to provide various types of information to a user in a visual or auditory manner while being mounted on a head of the user; and
an authentication server,
wherein the head mounted information processing device includes:
a display configured to display an image;
a microphone configured to collect a voice of the user and output an audio data signal;
a biometric authentication sensor configured to acquire tomographic data of the head of the user;
a controller configured to control the head mounted information processing device; and
a wireless communication interface configured to perform wireless communication with the authentication server,
wherein the controller transmits the tomographic data acquired by the biometric authentication sensor during an authentication period in which the user is uttering a passcode, to the authentication server through the wireless communication interface, and
wherein the authentication server collates the tomographic data with registration data of the user that is stored in advance and transmits an authentication result acquired by the collation to the head mounted information processing device.

8. The authentication system according to claim 7,
wherein the biometric authentication sensor includes a plurality of electrodes to acquire a distribution of electrical impedance in the head as the tomographic data.

9. The authentication system according to claim 7,
wherein the biometric authentication sensor includes a plurality of ultrasonic transducers to acquire an acoustic interference pattern in the head as the tomographic data.

10. The authentication system according to claim 7,
wherein the controller extracts a feature from the tomographic data and transmits data of the feature to the authentication server through the wireless communication interface.

11. The authentication system according to claim 7,
wherein the authentication server transmits the passcode to the head mounted information processing device, and
wherein, after causing the display to display the passcode from the authentication server, the controller detects the authentication period by performing voice recognition of the passcode uttered by the user from the audio data signal.

12. The authentication system according to claim 11,
wherein the controller transmits the audio data signal in the authentication period and the tomographic data to the authentication server through the wireless communication interface, and
wherein the authentication server performs voiceprint authentication based on the audio data signal and collates the tomographic data with the registration data.

13. An authentication method for authenticating a user with a head mounted information processing device configured to provide various types of information to the user in a visual or auditory manner while being mounted on a head of the user,
the head mounted information processing device including: a display configured to display an image; a microphone configured to collect a voice of the user and output an audio data signal; and a biometric authentication sensor configured to acquire tomographic data of the head of the user,
the authentication method comprising:
a first step of acquiring the tomographic data with the biometric authentication sensor during an authentication period in which the user is uttering a passcode; and
a second step of authenticating the user based on the tomographic data acquired by the first step.

14. The authentication method according to claim 13,
wherein the biometric authentication sensor includes a plurality of electrodes to acquire a distribution of electrical impedance in the head as the tomographic data.

15. The authentication method according to claim 13,
wherein the biometric authentication sensor includes a plurality of ultrasonic transducers to acquire an acoustic interference pattern in the head as the tomographic data.

16. The authentication method according to claim 13,
wherein the second step includes a step of collating the tomographic data with registration data of the user that is stored in advance.

17. The authentication method according to claim 13,
wherein the second step includes a step of extracting a feature from the tomographic data.

18. The authentication method according to claim 13, further comprising:
a third step of causing the display to display the passcode, the third step being executed before the first step; and
a fourth step of detecting the authentication period by performing voice recognition of the passcode uttered by the user from the audio data signal, the fourth step being executed after the third step.

19. The authentication method according to claim 18, further comprising a fifth step of performing voiceprint authentication based on the audio data signal in the authentication period.
